(19) United States
(12) Patent Application Publication
(10) Pub. No.: US 2003/0044441 A1
(43) Pub. Date: Mar. 6, 2003

(54) Cosmetic preparations containing pearly lustre waxes in the form of dispersed systems

(76) Inventors: Karl Heinz Schmid, Mettmann (DE); Claus Nieendick, Krefeld (DE); Anke Eggers, Duesseldorf (DE)

Correspondence Address: COGNIS CORPORATION, 2500 RENAISSANCE BLVD., SUITE 200, GULPH MILLS, PA, 19406

(21) Appl. No.: 10/204,156

(22) PCT Filed: Feb. 8, 2001
(86) PCT No.: PCT/EP01/01349

(30) Foreign Application Priority Data
Feb. 17, 2000 (DE) 100 07 322.0

(51) Int. Cl. A61K 7/42; A61K 7/32; A01N 25/00

(52) U.S. Cl. 424/401; 424/59; 424/405; 424/65

(57) ABSTRACT

Disclosed are cosmetic preparations that consist of a continuous phase and active agents dispersed therein and are characterized in that the continuous phase consists of pearly luster waxes.

### FIELD OF THE INVENTION

This invention relates to preparations containing active principles dispersed in a continuous, coherent phase.

### PRIOR ART

Besides the usual cosmetic ingredients, preparations used in cosmetics contain active principles such as, for example, biogenic agents, vitamins, deodorants, antidandruff agents, UV protection factors and the like. It is desirable to improve the stability of the active principles in cosmetic formulations by alternative supply forms. European patent EP 0 764 201 B1, for example, describes cleaning compositions containing peroxide compounds encapsulated in paraffin waxes as active principles.

Accordingly, the problem addressed by the present invention was to provide compositions which would enable a high concentration of active principles to be incorporated in cosmetic preparations by means of a carrier material and the stability of the active principles to be improved as a result. These compositions would ensure that the active principles are only released in use by mechanical, chemical or enzymatic action so that their action time would be extended and thus improved (for example in hair treatment preparations). In addition, the stability of highly unstable active principles in these compositions would be increased.

### DESCRIPTION OF THE INVENTION

The present invention relates to cosmetic preparations consisting of a continuous phase and active principles dispersed therein, characterized in that the continuous phase consists of pearlizing waxes.

It has surprisingly been found that active principles which are continuously dispersed in a pearlizing wax can be excellently incorporated in cosmetic preparations. Since the active principles are so to speak completely surrounded by the pearlizing wax, they can be dosed via the carrier material, i.e. the pearlizing wax, so that relatively high concentrations of active substances can be incorporated in cosmetic preparations. The release of the active principles is retarded by the pearlizing waxes and may require mechanical, thermal, chemical or enzymatic action. Another advantage in this regard is that the stability of, for example, highly unstable active principles is increased and their effectiveness in use can be improved by delayed release (for example hair treatment preparations and the like).

The present invention also relates to two processes for the production of cosmetic and/or pharmaceutical preparations in which pearlizing waxes and active principles are subjected to spray drying or spray crystallization either together or separately.

Pearlizing Waxes

Suitable pearlizing waxes are, for example, alkylene glycol esters; fatty acid alkanolamides; partial glycerides; esters of polybasic, optionally hydroxysubstituted carboxylic acids with fatty alcohols containing 6 to 22 carbon atoms; fatty compounds such as, for example, fatty alcohols, fatty ketones, fatty aldehydes, fatty ethers and fatty carbonates containing in all at least 24 carbon atoms; ring-opening products of olefin epoxides containing 12 to 22 carbon atoms with fatty alcohols containing 12 to 22 carbon atoms, fatty acids and/or polyols containing 2 to 15 carbon atoms and 2 to 10 hydroxyl groups; and mixtures thereof. In a preferred embodiment of the invention, the pearlizing waxes may be solid or liquid.

Alkylene glycol esters. The alkylene glycol esters are normally monoesters and/or diesters of alkylene glycols corresponding to formula (I):

R¹CO(OA)_{q}OR² (I)

in which R¹CO is a linear or branched, saturated or unsaturated acyl group containing 6 to 22 carbon atoms, R² is hydrogen or has the same meaning as R¹CO and A is a linear or branched alkylene group containing 2 to 4 carbon atoms and q is a number of 1 to 5. Typical examples are monoesters and/or diesters of ethylene glycol, propylene glycol, diethylene glycol, dipropylene glycol, triethylene glycol or tetraethylene glycol with fatty acids containing 6 to 22 and preferably 12 to 18 carbon atoms, such as caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof. Ethylene glycol monostearate and/or distearate is/are particularly preferred.

Fatty acid alkanolamides. Fatty acid alkanolamides which are suitable as pearlizing waxes correspond to formula (II):

R³CO—NR⁴—B—OH (II)

in which R³CO is a linear or branched, saturated or unsaturated acyl group containing 6 to 22 carbon atoms, R⁴ is hydrogen or an optionally hydroxysubstituted alkyl group containing 1 to 4 carbon atoms and B is a linear or branched alkylene group containing 1 to 4 carbon atoms. Typical examples are condensation products of ethanolamine, methyl ethanolamine, diethanolamine, propanolamine, methyl propanolamine and dipropanolamine and mixtures thereof with caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof. Stearic acid ethanolamide is particularly preferred.

Partial glycerides. Partial glycerides which have pearlizing properties are monoesters and/or diesters of glycerol with fatty acids, i.e. for example caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof. They correspond to formula (III):

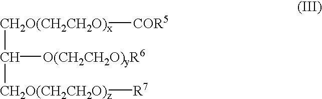

in which R⁵CO is a linear or branched acyl group containing 6 to 22 carbon atoms, R⁵ and R⁷ independently of one another represent hydrogen or have the same meaning as R⁷CO, x, y and z together stand for 0 or for a number of 1 to 30 and X is an alkali or alkaline earth metal, with the proviso that at least one of the two substituents R⁶ and R⁷ is hydrogen. Typical examples are lauric acid monoglyceride, lauric acid diglyceride, coconut oil fatty acid monoglyceride, coconut fatty acid triglyceride, palmitic acid monoglyceride, palmitic acid triglyceride, stearic acid monoglyceride, stearic acid diglyceride, isostearic acid monoglyceride, isostearic acid diglyceride, oleic acid monoglyceride, oleic acid diglyceride, tallow fatty acid monoglyceride, tallow fatty acid diglyceride, behenic acid monoglyceride, behenic acid diglyceride, erucic acid monoglyceride, erucic acid diglyceride and technical mixtures thereof which may still contain small quantities of triglyceride from the production process.

Polybasic carboxylic acid and hydroxycarboxylic acid esters. Other suitable pearlizing waxes are esters of polybasic, optionally hydroxysubstituted carboxylic acids with fatty alcohols containing 6 to 22 carbon atoms. The acid component of these esters may be selected, for example, from malonic acid, maleic acid, fumaric acid, adipic acid, sebacic acid, azelaic acid, dodecanedioic acid, phthalic acid, isophthalic acid and, more particularly, succinic acid and also malic acid, citric acid and, more particularly, tartaric acid and mixtures thereof. The fatty alcohols contain 6 to 22, preferably 12 to 18 and more preferably 16 to 18 carbon atoms in the alkyl chain. Typical examples are caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. The esters may be present as full or partial esters; monoesters and, above all, diesters of carboxylic or hydroxycarboxylic acids preferably being used. Typical examples are succinic acid mono- and dilauryl ester, succinic acid mono- and dicetearyl ester, succinic acid mono- and distearyl ester, tartaric acid mono- and dilauryl ester, tartaric acid mono- and dicocoalkyl ester, tartaric acid mono- and dicetearyl ester, citric acid mono-, di- and trilauryl ester, citric acid mono-, di- and tricocoalkyl ester and citric acid mono-, di- and tricetearyl ester.

Fatty alcohols. Another group of pearlizing waxes are long-chain fatty alcohols corresponding to formula (IV):

R⁸OH (IV)

in which R⁸ is a linear alkyl group containing 24 to 48 and preferably 32 to 36 carbon atoms. The substances mentioned are generally oxidation products of long-chain paraffins.

Fatty ketones. Fatty ketones suitable as component (a) preferably correspond to formula (V):

R⁹—CO—R¹⁰ (V)

in which R⁹ and R¹⁰ independently of one another represent alkyl and/or alkenyl groups containing 1 to 22 carbon atoms, with the proviso that they contain a total of at least 24 and preferably 32 to 48 carbon atoms. The ketones may be prepared by known methods, for example by pyrolysis of the corresponding fatty acid magnesium salts. The ketones may be symmetrical or non-symmetrical, although the two substituents R⁹ and R¹⁰ preferably differ from one another by only one carbon atom and are derived from fatty acids containing 16 to 22 carbon atoms. Stearone is distinguished by particularly advantageous pearlizing properties.

Fatty aldehydes. Fatty aldehydes suitable as pearlizing waxes correspond to formula (VI):

R¹¹COH (VI)

in which R¹¹CO is a linear or branched acyl group containing 24 to 48 and preferably 28 to 32 carbon atoms.

Fatty ethers. Other suitable pearlizing waxes are fatty ethers corresponding to formula (VII):

R¹²—O—R¹³ (VII)

in which R¹² and R¹³ independently of one another represent alkyl and/or alkenyl groups containing 1 to 22 carbon atoms, with the proviso that they contain a total of at least 24 and preferably 32 to 48 carbon atoms. Fatty ethers of the type mentioned are normally prepared by acidic condensation of the corresponding fatty alcohols. Fatty ethers with particularly advantageous pearlizing properties are obtained by condensation of fatty alcohols containing 16 to 22 carbon atoms such as, for example, cetyl alcohol, cetearyl alcohol, stearyl alcohol, isostearyl alcohol, oleyl alcohol, behenyl alcohol and/or erucyl alcohol.

Fatty carbonates. Component (a) may also be selected from fatty carbonates corresponding to formula (VIII):

R¹⁴O—CO—OR¹⁵ (VIII)

in which R¹⁴ and R¹⁵ independently of one another are alkyl and/or alkenyl groups containing 1 to 22 carbon atoms, with the proviso that they contain a total of at least 24 and preferably 32 to 48 carbon atoms. The substances are obtained by transesterifying dimethyl or diethyl carbonate, for example, with the corresponding fatty alcohols by methods known per se. Accordingly, the fatty carbonates may be symmetrical or non-symmetrical. However, carbonates in which R¹⁴ and R¹⁵ are the same and represent alkyl groups containing 16 to 22 carbon atoms are preferably used. Transesterification products of dimethyl or diethyl carbonate with cetyl alcohol, cetearyl alcohol, stearyl alcohol, isostearyl alcohol, oleyl alcohol, behenyl alcohol and/or erucyl alcohol in the form of their monoesters and diesters and technical mixtures thereof are particularly preferred.

Fatty acids. Other suitable pearlizing waxes are aliphatic, optionally hydroxy-substituted carboxylic acids containing 16 to 30 carbon atoms such as, for example, stearic acid, cetyl stearic acid, hydroxystearic acid and behenic acid and technical mixtures thereof.

Epoxide ring-opening products. The ring-opening products are known substances which are normally obtained by acid-catalyzed reaction of terminal or internal olefin epoxides with aliphatic alcohols. The reaction products preferably correspond to formula (IX):

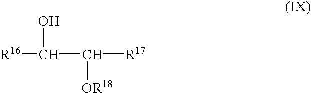

in which R¹⁶ and R¹⁷ represent hydrogen or an alkyl group containing 10 to 20 carbon atoms, with the proviso that the sum total of carbon atoms of R¹⁶ and R¹⁷ is between 10 and 20 and R¹⁸ is an alkyl and/or alkenyl group containing 12 to 22 carbon atoms and/or the residue of a polyol containing 2 to 15 carbon atoms and 2 to 10 hydroxyl groups. Typical examples are ring-opening products of α-dodecene epoxide, α-hexadecene epoxide, α-octadecene epoxide, α-eicosene epoxide, α-docosene epoxide, i-dodecene epoxide, i-hexadecene epoxide, i-octadecene epoxide, i-eicosene epoxide and/or i-docosene epoxide with lauryl alcohol, cocofatty alcohol, myristyl alcohol, cetyl alcohol, cetearyl alcohol, stearyl alcohol, isostearyl alcohol, oleyl alcohol, elaidyl alcohol, petroselinyl alcohol, linolyl alcohol, linolenyl alcohol, behenyl alcohol and/or erucyl alcohol. Ring opening products of hexa- and/or octadecene epoxides with fatty alcohols containing 16 to 18 carbon atoms are preferably used. If polyols are used instead of the fatty alcohols for the ring opening reaction, they are selected for example from the following substances: glycerol; alkylene glycols such as, for example, ethylene glycol, diethylene glycol, propylene glycol, butylene glycol, hexylene glycol and polyethylene glycols with an average molecular weight of 100 to 1,000 dalton; technical oligoglycerol mixtures with a degree of self-condensation of 1.5 to 10 such as, for example, technical diglycerol mixtures with a diglycerol content of 40 to 50% by weight; methylol compounds such as, in particular, trimethylol ethane, trimethylol propane, trimethylol butane, pentaerythritol and dipentaerythritol; lower alkyl glucosides, more particularly those containing 1 to 8 carbon atoms in the alkyl chain such as, for example, methyl and butyl glucoside; sugar alcohols containing 5 to 12 carbon atoms such as, for example, sorbitol or mannitol, sugars containing 5 to 12 carbon atoms such as, for example, glucose or sucrose; amino sugars such as, for example, glucamine. The preparations according to the invention may contain the pearlizing waxes in quantities of 50 to 99.9, preferably 55 to 90 and more particularly 60 to 85% by weight, based on the preparations.

Active Principles for Cosmetic and/or Pharmaceutical Applications

Typical examples of active principles used in cosmetic and pharmaceutical preparations are surfactants, cosmetic oils, stabilizers, biogenic agents, vitamins, deodorants, antiperspirants, antidandruff agents, UV protection factors, antioxidants, preservatives, insect repellents, self-tanning agents, tyrosine inhibitors (depigmenting agents), perfume oils and dyes. These active principles may be present in quantities of 0.1 to 50% by weight, preferably 10 to 45% by weight and more particularly 15 to 40% by weight, based on the preparations.

Anionic, nonionic, cationic and/or amphoteric or zwitterionic surfactants may be encapsulated as surfactants. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl(ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217.

Suitable cosmetic oil components are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Metal salts of fatty acids such as, for example, magnesium, aluminum and/or zinc stearate or ricinoleate may be used as stabilizers.

In the context of the invention, biogenic agents are, for example, tocopherol, tocopherol acetate, tocopherol palmitate, ascorbic acid, deoxyribonucleic acid, retinol, bisabolol, allantoin, phytantriol, panthenol, AHA acids, koji acid, amino acids, ceramides, pseudoceramides, essential oils, plant extracts and vitamin complexes.

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monolaurate (GML), diglycerol monocaprate (DMC), salicylic acid-N-alkylamides such as, for example, salicylic acid-n-octyl amide or salicylic acid-n-decyl amide.

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT, Henkel KGaA, Dusseldorf, FRG). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of labdanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

non-aqueous solvents such as, for example, ethanol, propylene glycol and/or glycerol.

Suitable astringent active principles of antiperspirants are, above all, salts of aluminum, zirconium or zinc. Suitable antihydrotic agents of this type are, for example, aluminum chloride, aluminum chlorohydrate, aluminum dichlorohydrate, aluminum sesquichlorohydrate and complex compounds thereof, for example with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, for example with amino acids, such as glycine.

Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH regulators, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Suitable antidandruff agents are climbazol, octopirox, ketoconazole and zinc pyrithione.

Examples of UV protection factors include organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor, as described in EP 0693471 B1;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzophenone, 2-hydroxy-4-methoxy-4′-methylbenzophenone, 2,2′-dihydroxy -4-methoxybenzophenone;

esters of benzalmalonic acid, preferably 4-methoxybenzalmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone, as described in EP 0 818 450 A1, or Dioctyl Butamido Triazine (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives, as described in EP 0 694 521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example 1-(4′-tert.butylphenyl)-3-(4′-methoxyphenyl)-propane-1,3-dione, 4-tert-butyl-4′-methoxydibenzoylmethane (Parsol 1789), 1-phenyl-3-(4′-isopropylphenyl)-propane-1,3-dione and the eneamine compounds described in DE 19712033 A1 (BASF). The UV-A and UV-B filters may of course also be used in the form of mixtures. Besides the soluble substances mentioned, insoluble pigments, i.e. finely dispersed metal oxides or salts, may also be used for this purpose. Examples of suitable metal oxides are, in particular, zinc oxide and titanium dioxide and also oxides of iron, zirconium, silicon, manganese, aluminum and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have an average diameter of less than 100 nm, preferably from 5 to 50 nm and more preferably from 15 to 30 nm. They may be spherical in shape although ellipsoidal particles or other non-spherical particles may also be used. The pigments may also be surface-treated, i.e. hydrophilicized or hydrophobicized. Typical examples are coated titanium dioxides such as, for example, Titandioxid T 805 (Degussa) or Eusolex® T2000 (Merck). Suitable hydrophobic coating materials are, above all, silicones and particularly trialkoxyoctyl silanes or simethicones. So-called micro- or nanopigments are preferably used in sun protection products. Micronized zinc oxide is preferably used. Other suitable UV filters can be found in P. Finkel's review in SÖFW-Journal 122, 543 (1996).

Besides the two above-mentioned groups of primary protection factors, secondary protection factors of the antioxidant type may also be used. Secondary sun protection factors of the antioxidant type interrupt the photochemical reaction chain which is initiated when UV rays penetrate into the skin. Typical examples of suitable antioxidants are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmol to lmol/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”). Suitable insect repellents are N,N-diethyl-m-toluamide, pentane-1,2-diol or Ethyl Butylacetylaminopropionate. A suitable self-tanning agent is dihydroxyacetone. Suitable tyrosine inhibitors which prevent the formation of melanin and are used in depigmenting agents are, for example, arbutin, koji acid, coumaric acid and ascorbic acid (vitamin C).

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamon, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable dyes are any of the substances suitable and approved for cosmetic purposes as listed, for example, in the publication “Kosmetische Färbemittel” of the Farbstoffkommission der Deutschen Forschungsgemeinschaft, Verlag Chemie, Weinheim, 1984, pages 81 to 106. These active principles may also be present in the capsules for aesthetic reasons only and are not intended for controlled release.

Spray Drying or Spray Crystallization

The dryer into which the pearlizing waxes and active principles are sprayed can be any type of dryer. In one preferred embodiment of the process, drying is carried out by spray drying or spray crystallization in a drying tower, more particularly under reduced pressure. In this case, the aqueous preparations are exposed in known manner to a stream of drying gas in fine-particle form. Applicants describe an embodiment of spray drying using superheated steam in a number of published patents. The operating principle disclosed in those publications is hereby specifically included as part of the disclosure of the present invention. Reference is made in particular to the following publications: DE 40 30 688 A1 and the further developments according to DE 42 04 035 A1; DE42 04 090 A1; DE4206050A1; DE4206521 A1; DE4206495A1; DE4208773A1; DE 42 09 432 A1 and DE 42 34 376 A1. This process was introduced in connection with the production of the defoamer granules.

The pearlizing waxes and active principles may be subjected to spray drying or spray crystallization together. The active principles may also be subjected to spray drying or spray crystallization on their own and then charged with the pearlizing waxes in a spray mixer. In this case, a pearlizing wax/wax mixture is normally sprayed in liquid form, i.e. about 5 to 10° C. above its melting point, onto a matrix containing the active principle. The matrix containing the active principle is present as a solid and may consist of hydrophilic waxes, crystallizing surfactant preparations or carrier materials binding the active principle with which they are compatible. The ratio by weight of pearlizing wax to active principle is 10:1 to 1:1 and preferably 7:1 to 2:1. Particle sizes of 3 to 100, preferably 4 to 50 and more particularly 5 to 25 μm are obtained. In a preferred embodiment of the invention, spray drying or spray crystallization may be carried out using a double tube, the pearlizing waxes being introduced through the outer tube and the active principles through the inner tube. In this way, the active principles can form an inner core which is surrounded by the pearlizing waxes in a continuous phase. By continuous phase is meant a homogeneous phase which remains stable, i.e. unchanged, even over prolonged periods of storage.

Cosmetic and/or Pharmaceutical Preparations

The compositions according to the invention may be used for the production of cosmetic and/or pharmaceutical preparations such as, for example; hair shampoos, oral and dental care preparations, hair lotions, foam baths, shower baths, creams, gels, lotions, alcoholic and aqueous/alcoholic solutions, emulsions, wax/fat compounds, stick preparations or ointments. The compositions according to the invention may be present in the preparations in quantities of 0.01 to 30, preferably 0.5 to 20 and more particularly 1 to 10% by weight, based on the active substance content. They may contain mild surfactants, oil components, emulsifiers, superfatting agents, consistency factors, thickeners, polymers, silicone compounds, fats, waxes, stabilizers, biogenic agents, deodorizers, antiperspirants, antidandruff agents, film formers, swelling agents, UV protection factors, antioxidants, hydrotropes, preservatives, insect repellents, self-tanning agents, solubilizers, perfume oils, dyes and the like as further auxiliaries and additives. Many of these auxiliaries are discussed in detail in earlier chapters and need not be discussed again here.

Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines and/or protein fatty acid condensates, preferably based on wheat proteins.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2 252 840 A and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar® CBS, Jaguar® C-17, Jaguar® C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Typical examples of fats are glycerides while suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes, microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

The total percentage content of auxiliaries and additives may be from 1 to 50% by weight and is preferably from 5 to 40% by weight, based on the particular composition. The compositions may be produced by standard hot or cold processes and are preferably produced by the phase inversion temperature method.

### EXAMPLES

The cosmetic preparations which consist of a continuous phase and an active principle dispersed therein (compounds 1 to 6) were produced by spray crystallization of the active principles listed below together with the pearlizing wax mixtures. Compounds 1 to 6 have the following composition:

1) Dipalmitoylethyl Hydroxyethylmonium Methosulfate:Cetearyl Alcohol:Ethylene Glycol Distearate (25:20:55)

2) Uvasorb:Cetearyl Alcohol:Ethylene Glycol Distearate (40:15:45)

3) Ceramide 3:Distearylether:Cetearyl Alcohol (25:50:25)

4) Ceramide 3:Ethylene Glycol Distearate (25:75)

5) Lactic acid:Ethylene Glycol Monostearate:Cetearyl Alcohol (25:50:25)

6) Alpha Tocopherol I:Ethylene Glycol Monostearate:Cetearyl Alcohol:Glycerol Monostearate (20:40:20:20)

The following Tables contain a few selected examples of cosmetic preparations in which the compounds according to the invention are used (Example 1 in each Table). For comparison (C1), preparations were produced without these compounds. The appearance of the preparations was evaluated along with storage stability over a period of 4 weeks at 20° C. (+=stable; −=unstable; separates/sediments, agglomerates).

TABLE 1
Conditioning shampoo-quantities in % by weight active substance

| Composition | C1 | 1 | 2 |
|---|---|---|---|
| Sodium Laureth Sulfate | 10 | 10 | 10 |
| Cocamidopropyl Betaine | 3 | 3 | 3 |
| Dipalmitoylethyl | 0.5 | — | — |
| Hydroxyethylmonium | | | |
| Methosulfate | | | |
| Cetearyl Alcohol | 0.4 | 0.3 | 0.3 |
| Ethylene Glycol Distearate | 1.1 | — | — |
| Compound 1 | — | 2 | 4 |
| Water | to 100 | | |
| Appearance | Opaque/ | Opaque/ | Opaque/ |
| | pearlescent | pearlescent | pearlescent |
| Storage stability | − | + | + |

TABLE 2
Shampoo containing oil-soluble UV filter-quantities
in % by weight active substance

| Composition | C1 | 1 | 2 |
|---|---|---|---|
| Sodium Laureth Sulfate | 10 | 10 | 10 |
| Cocamidopropyl Betaine | 2 | 2 | 2 |
| Cocoglucoside | 2 | 2 | 2 |
| Uvasorb ® HEB | 0.8 | — | — |
| Cetearyl Alcohol | 0.3 | — | — |
| Ethylene glycol distearate | 0.9 | — | — |
| Compound 2 | — | 2 | 4 |
| Water | to 100 | | |
| Appearance | Opaque/ | Opaque/ | Opaque/ |
| | pearlescent | pearlescent | pearlescent |
| Storage stability | − | + | + |

TABLE 3
Care shampoo containing Ceramide 3-quantities
in % by weight active substance

| Composition | C1 | 1 | 2 |
|---|---|---|---|
| Sodium Laureth Sulfate | 10 | 10 | 10 |
| Cocamidopropyl Betaine | 4 | 4 | 4 |
| Cocoglucoside | 2 | 2 | 2 |
| D-panthenol | 0.3 | 0.3 | 0.3 |
| Ceramide 3 | 0.5 | — | — |
| Distearylether | 1 | — | — |
| Cetearyl Alcohol | 1 | — | — |
| Compound 3 | — | 2.5 | 5 |
| Water | to 100 | | |
| Appearance | Opaque/ | Opaque/ | Opaque/ |
| | pearlescent | pearlescent | pearlescent |
| Storage stability | − | + | + |

TABLE 4
Rinse containing Ceramide 3-quantities in % by weight active substance

| Composition | C1 | 1 | 2 |
|---|---|---|---|
| Sodium Laureth Sulfate | 15 | 15 | 15 |
| Cocamidopropyl Betaine | 4 | 4 | 4 |
| Cocoglucoside | 6 | 6 | 6 |
| Aloe Vera | 0.1 | 0.1 | 0.1 |
| Ceramide 3 | 0.5 | — | — |
| Ethylene Glycol Distearate | 1.5 | — | — |
| Compound 4 | — | 2 | 4 |
| Glycerol | 4 | 4 | 4 |
| Ethanol | 4 | 4 | 4 |
| Water | to 100 | | |
| Appearance | Opaque/ | Opaque/ | Opaque/ |
| | pearlescent | pearlescent | pearlescent |
| Storage stability | − | + | + |

TABLE 5
Care shampoo containing lactic acid-quantities
in % by weight active substance

| Composition | C1 | 1 | 2 |
|---|---|---|---|
| Sodium Laureth Sulfate | 9 | 9 | 9 |
| Cocamidopropyl Betaine | 3 | 3 | 3 |
| Cocoglucoside | 4 | 4 | 4 |
| D-panthenol | 0.3 | 0.3 | 0.3 |
| Lactic acid | 0.5 | — | — |
| Ethylene Glycol Distearate | 1 | — | — |
| Cetearyl Alcohol | 0.5 | — | — |
| Compound 5 | — | 2 | 4 |
| Water | to 100 | | |
| Appearance | Opaque/ | Opaque/ | Opaque/ |
| | pearlescent | pearlescent | pearlescent |
| Storage stability | − | + | + |

TABLE 6
Care shampoo containing vitamin E-quantities
in % by weight active substance

| Composition | C1 | 1 | 2 |
|---|---|---|---|
| Sodium Laureth Sulfate | 8 | 8 | 8 |
| Cocamidopropyl Betaine | 3 | 3 | 3 |
| Cocoglucoside | 4 | 4 | 4 |
| D-panthenol | 0.3 | 0.3 | 0.3 |
| Alpha Tocopherol | 1 | — | — |
| Ethylene Glycol Distearate | 2 | — | — |
| Cetearyl Alcohol | 1 | — | — |
| Glycerin Monostearate | 1 | — | — |
| Compound 6 | — | 4 | 8 |
| Water | to 100 | | |
| Appearance | Opaque/ | Opaque/ | Opaque/ |
| | pearlescent | pearlescent | pearlescent |
| Storage stability | − | + | + |

1. Cosmetic preparations consisting of a continuous phase and active principles dispersed therein, characterized in that the continuous phase consists of pearlizing waxes.

2. Preparations as claimed in claim 1, characterized in that they contain pearlizing waxes selected from the group consisting of alkylene glycol esters, fatty acid alkanolamides, partial glycerides, esters of polybasic, optionally hydroxysubstituted carboxylic acids with fatty alcohols containing 6 to 22 carbon atoms, fatty alcohols, fatty ketones, fatty aldehydes, fatty ethers and/or fatty carbonates containing in all at least 24 carbon atoms, fatty acids and hydroxyfatty acids containing 16 to 30 carbon atoms and ring-opening products of olefin epoxides containing 12 to 22 carbon atoms with fatty alcohols containing 12 to 22 carbon atoms and/or polyols containing 2 to 15 carbon atoms and 2 to 10 hydroxyl groups.

3. Preparations as claimed in claims 1 and/or 2, characterized in that they contain solid or liquid pearlizing waxes.

4. Preparations as claimed in at least one of claims 1 to 3, characterized in that they contain active principles selected from the group consisting of surfactants, cosmetic oils, stabilizers, biogenic agents, vitamins, deodorants, antiperspirants, antidandruff agents, UV protection factors, antioxidants, preservatives, insect repellents, self-tanning agents, perfume oils, aromas and dyes.

5. Preparations as claimed in at least one of claims 1 to 4, characterized in that the active principles form an inner core which is surrounded by the continuous phase.

6. A process for the production of cosmetic and/or pharmaceutical preparations in which the active principles are subjected to spray drying or spray crystallization and then charged with the pearlizing wax.

7. A process for the production of cosmetic and/or pharmaceutical preparations in which a mixture of pearlizing waxes and active principles is subjected to spray drying or spray crystallization.

8. A process as claimed in claim 6, characterized in that spray drying or spray crystallization is carried out using a double tube in which the pearlizing waxes are introduced through an outer tube and the active principles through an inner tube.

9. A process as claimed in at least one of claims 6 to 8, characterized in that spray drying or spray crystallization is carried out under reduced pressure.

10. The use of the preparations claimed in claim 1 for the production of cosmetic and/or pharmaceutical preparations.
